(19) United States
(12) Patent Application Publication
(10) Pub. No.: US 2024/0001025 A1
(43) Pub. Date: Jan. 4, 2024

(54) HUB FOR REUSABLE DRUG-DELIVERY DEVICE

(71) Applicant: Eli Lilly and Company, Indianapolis, IN (US)

(72) Inventors: Alexander Joseph BAKER, Somerville, MA (US); Kevin Harrison DUFFY, Pittsboro, IN (US); Kaitlyn Mary EDDY, Norwood, MA (US); Dae Il KWON, Shoreline, WA (US); Labi SHRESTHA, Somerville, MA (US); Rhea SIRKAR, Boston, MA (US)

(21) Appl. No.: 18/258,018

(22) PCT Filed: Jan. 5, 2022
(86) PCT No.: PCT/US2022/011236

§ 371 Date: Jun. 16, 2023

Related U.S. Application Data
(60) Provisional application No. 63/134,984, filed on Jan. 8, 2021.

(51) Int. Cl. A61M 5/00

(52) U.S. Cl. CPC A61M 5/002; A61M 2005/005

(57) ABSTRACT

A recharging and medication storage hub for a re-usable drug-delivery device is provided. According to some embodiments, the hub comprises a housing defining a port sized to receive at least part of the re-usable drug-delivery device, a charging station disposed on the housing configured to charge a rechargeable power source mounted on the drug-delivery device, and a medication storage chamber disposed in alignment with the port, the storage chamber configured to store a single-use medication cartridge. When at least part of the drug-delivery device is inserted through the port and into the medication storage chamber, a coupling mechanism is configured to releasably couple with and withdraw the medication cartridge from the medication storage chamber. In some embodiments, the hub may further comprise a temperature-changing device configured to control an interior temperature of the medication storage chamber.

### FIELD OF THE DISCLOSURE

The present disclosure relates to a hub for holding a reusable drug-delivery device. More particularly, the present disclosure relates to a hub for recharging a reusable drug-delivery device, and for storing medication cartridges configured to be used by the reusable drug-delivery device.

### BACKGROUND OF THE DISCLOSURE

Re-usable drug delivery devices are widely used by medical professionals and by those who self-medicate. Such devices may be configured to receive disposable, single-use medication cartridges, and to administer medication stored within such cartridges to a patient via an injection. However, the process for replacing a spent medication cartridge with a new cartridge for such re-usable drug-delivery devices can be manually laborious or difficult, particularly for users with low manual dexterity. Furthermore, users of re-usable drug-delivery devices generally need a way to store and/or organize the medication cartridges used by re-usable drug-delivery devices. Some types of medication cartridges may need to be stored at a storage temperature (e.g., refrigerated at 32-35 degrees Fahrenheit) to prevent spoliation, and then warmed up to an administration temperature (e.g., brought up to room temperature) just before administration.

### SUMMARY

According to an exemplary embodiment of the present disclosure, a recharging and medication storage hub for a drug-delivery device is provided, the hub comprising: a housing defining a port sized to receive at least part of the drug-delivery device; a charging station disposed on the housing configured to charge a rechargeable power source mounted on the drug-delivery device when at least part of the drug-delivery device is positioned in proximity with the charging station; and a medication storage chamber configured to align with the port, the medication storage chamber configured to store a medication cartridge; wherein when at least part of the drug-delivery device is inserted through the port and into the medication storage chamber and then withdrawn from the medication storage chamber, the medication storage chamber is configured to allow the drug-delivery device to withdraw the medication cartridge from the medication storage chamber.

According to another embodiment of the present disclosure, a method for using a drug-delivery device and a recharging and medication storage hub for the drug-delivery device is provided. The hub may have a port, a charging station, a medication storage chamber in alignment with the port, and a medication cartridge storing a medication disposed within the medication storage chamber. The method may comprise placing at least part of the drug-delivery device in proximity with the charging station to charge a rechargeable power source mounted on the drug-delivery device; removing the drug-delivery device from the charging station; inserting at least part of the drug-delivery device through the port and into the medication storage chamber to couple the drug-delivery device with the medication cartridge stored within the medication storage chamber; and removing the drug delivery device from the port once coupled with the medication cartridge so as to remove the medication cartridge from the medication storage chamber.

According to yet another embodiment of the present disclosure, a method for using a recharging and medication storage hub system in conjunction with a mobile device is provided. The method may comprise providing the hub system, the hub system comprising: a drug-delivery device having a rechargeable power source, and a hub, the hub comprising: a port having a door configured to at least one of (i) move between an open and a closed position and (ii) switch between a locked and an unlocked configuration, a charging station configured to charge the rechargeable power source of the drug-delivery device when at least a portion of the drug-delivery device is placed in proximity with the charging station, a medication storage chamber, and a medication cartridge storing a medication disposed within the medication storage chamber. The method may further comprise establishing a communication link between the hub and the mobile device; sending, from the mobile device, an instruction to the hub to prepare the medication cartridge for administration, wherein the instruction causes the hub to move the door to the open position or switch the door to the unlocked configuration to allow access to the medication cartridge; removing the drug-delivery device from the charging station; inserting the drug-delivery device into the port to couple the drug-delivery device with the medication cartridge; and removing the drug-delivery device from the port once coupled with the medication cartridge so as to remove the medication cartridge from the medication storage chamber.

Among other advantages, one exemplary advantage of the disclosed hub is that it provides a single, convenient, integrated hub that is configured to both recharge a drug-delivery device and store medication cartridges for use with the drug-delivery device. Another exemplary advantage of some embodiments is that the hub may include an integrated temperature-changing device that maintains stored medication cartridges at a storage temperature, thereby relieving users from providing separate cold storage for medication cartridges. Another exemplary advantage of some embodiments is that the hub may use the temperature-changing device to warm the cartridges to an administration temperature just before administration. In some embodiments, the temperature-changing device may bring the cartridge to an administration temperature faster than if the cartridge were allowed to warm up to room temperature by itself. Another exemplary advantage of some embodiments is that the hub keeps stored medication cartridges secure by locking the cartridges behind a closed port—the port may not open until the user provides an instruction from the user's mobile device. Yet another exemplary advantage of some embodiments is that the hub may serve as a sharps container for disposing of used medication cartridges, thus relieving the user of the need to provide a separate sharps container. Other advantages will be recognized by those of ordinary skill in the art.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a conceptual block diagram of an exemplary hub for holding an exemplary drug-delivery device, and a drug-delivery device and mobile device for use with the hub, according to some embodiments.

FIG. 2 is a perspective view of a first embodiment of the hub for holding the drug-delivery device.

FIG. 3 is a perspective view of the interior of the first embodiment of the hub.

FIG. 4 is a top perspective view of a first embodiment of a medication bin configured to be inserted into the hub.

FIG. 5 is a bottom perspective view of the first embodiment of the medication bin.

FIG. 6 is a profile view of an exemplary medication cartridge.

FIG. 7 and FIG. 8 depict exemplary perspective views of the first embodiment of the hub and drug-delivery device in operation.

FIG. 9 is a top-down view of the medication bin, illustrating how the bin may be rotated to move an exhausted medication cartridge out of the way and position a fresh medication cartridge for use.

FIG. 10 is a perspective view of a second embodiment of a hub for holding a drug-delivery device.

FIG. 11 is a perspective view of the second embodiment of the hub in which a medication bin has been removed.

FIG. 12 is a profile view of an exemplary coupling mechanism within a drug-delivery device for releasably coupling with a medication cartridge.

FIG. 13 is a perspective view of the exemplary coupling mechanism.

FIG. 14 is a flow-chart illustrating an exemplary process for using a recharging and medication storage hub.

FIG. 15 is a flow-chart illustrating an exemplary process for using a recharging and medication with a mobile device.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

The term “logic,” “control logic”, “application”, “method”, or “process” as used herein may include software and/or firmware executing on one or more programmable processors, application-specific integrated circuits (ASICs), field-programmable gate arrays (FPGAs), digital signal processors (DSPs), hardwired logic, or combinations thereof. Therefore, in accordance with the embodiments, various logic may be implemented in any appropriate fashion and would remain in accordance with the embodiments herein disclosed.

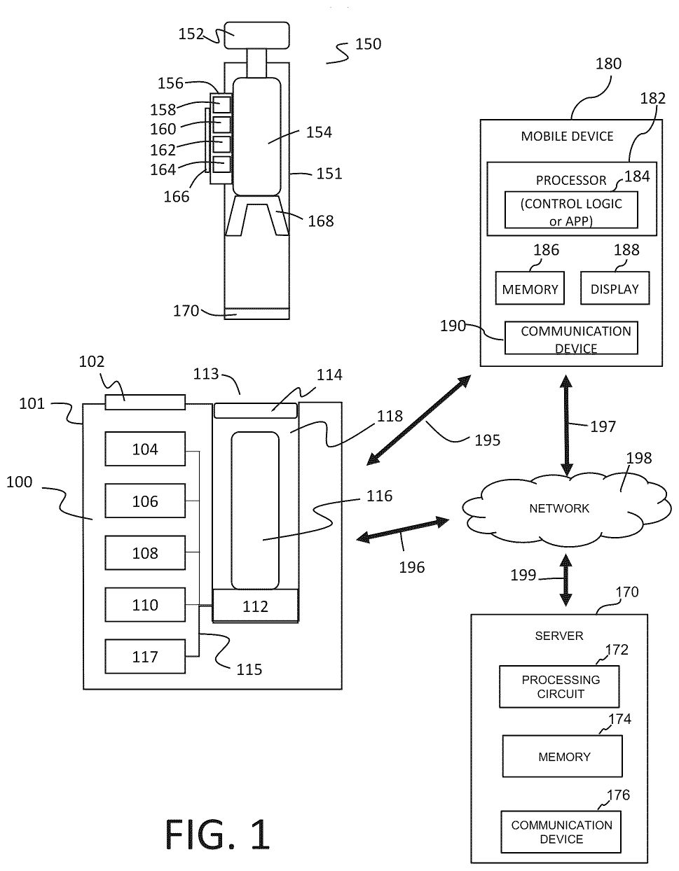

FIG. 1 depicts a block diagram of an exemplary hub 100 for use with an exemplary drug-delivery device 150, in accordance with some embodiments. Hub 100 includes a housing 101 that houses or supports the components of the hub 100. Housing 101 defines a port 113 that is covered by a movable door 114. Door 114 may be removed entirely from housing 101 or slid or swung out of the way to provide access to a medication storage chamber 118.

Medication storage chamber 118 is configured to hold or store a cartridge 116 that has been pre-filled with medication. Chamber 118 is aligned with port 113 such that the chamber 118 may be accessed through port 113. As used herein, the term “medication” or “drug” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of delivery by the above device. The medication as used in the device may be formulated with one or more excipients.

Cartridge 116 may be configured to be inserted into or coupled with reusable delivery device 150. Once loaded or coupled with cartridge 116, reusable delivery device 150 may be operated by a patient, caregiver or healthcare professional to deliver the medication within cartridge 116 to a person, e.g., via an injection, a spray, an intravenous feed, or other mechanism. Cartridge 116 may be a single-use cartridge that is designed to be decoupled from reusable delivery device once its store of medication is exhausted; once decoupled, cartridge 116 may be discarded. In some embodiments, reusable delivery device 150 may be configured to deliver all the medication stored within cartridge 116 in a single dose. In other embodiments, reusable delivery device 150 may be configured to deliver the medication stored within cartridge 116 over multiple doses.

Medication storage chamber 118 may also include a temperature-changing device 112. Temperature-changing device may include a cooling device configured to lower an interior temperature of medication storage chamber 118 (and thereby lower the temperature of cartridge 116 stored within chamber 118) to preserve medication stored within cartridge 116 while it is being held in storage. One example of a suitable cooling device is a Peltier device that transfers heat from one side of the device to the other using electrical energy. Alternatively, temperature-changing device 112 may cool the interior of medication storage chamber 118 using a vapor-compression refrigeration process, and/or using circulation of coolant. Temperature-changing device 112 may also be configured to heat the interior of medication storage chamber 118 when it is time to administer the medication within cartridge 116, e.g., to raise the medication stored within cartridge 116 to room temperature or body temperature. Exemplary heating devices include Peltier devices, or electrical heating coils or wires. In some embodiments, a single component or a single type of component (e.g., a Peltier device) may be used to both cool and heat the medication storage chamber.

Hub 100 also includes a charging station 102. Charging station 102 may be coupled with a source of electrical power, such as a battery or a wall power outlet (not shown). When a charging contact 170 on drug-delivery device 150 is placed in contact or close proximity with charging station 102, charging station 102 may be configured to recharge a rechargeable power source 158 (e.g., a battery or super capacitor) on device 150 either directly or inductively. As used herein, a drug-delivery device may be considered in “proximity” with charging station 102 when the distance between charging station 102 and the drug-delivery device is small enough to allow charging station 102 to inductively or conductively charge the rechargeable power source on the drug-delivery device. Charging station 102 may comprise, or be disposed within, a receptable configured to receive all or part of device 150. Alternatively, or in addition, charging station 102 may comprise a visually distinguishable portion of an exterior surface of hub 100, and/or charging station 102 may comprise a protrusion from the exterior surface of hub 100 that is configured to fit into a corresponding receptable on reusable drug-delivery device 150. In some embodiments, charging station 102 may also comprise a wire that extends from an exterior surface of hub 100, and which is configured to plug into a corresponding port on drug-delivery device 150.

Hub 100 further includes a controller 104 communicably coupled with a user-interface 106, a cartridge sensor 108, one or more additional sensors 117, and/or a communication device 110, via an internal bus 115. Controller 104 may include at least one processor that executes software and/or firmware stored in memory (not shown) of hub 100. The software/firmware code contains instructions that, when executed by the processor of controller 104, causes controller 104 to perform the functions described herein. In some embodiments, either alternatively or in addition, controller 104 may include any processing circuit that receives and processes data signals, and which outputs results in the form of one or more electrical signals as a result. Such processing circuits may include one or more Application Specific Integrated Circuits (ASICs), field-programmable gate arrays (FPGAs), digital signal processors (DSPs), hardwired logic, or any combination thereof. User-interface 106 may comprise a screen, lights (e.g., LEDs), and/or speakers for communicating information to a user of hub 100, such as a status of hub 100 and/or reusable drug-delivery device 150. User-interface 106 may also comprise a touch-sensitive screen, buttons, sliders, and/or dials/knobs for receiving user-input from a user of hub 100.

Cartridge sensor 108 is a sensor that identifies or senses a presence of medication cartridge 116. Cartridge sensor 108 may comprise a visual or optical sensor that senses a presence of medication cartridge 116 or a presence of a container holding cartridge 116, or identifies said cartridge, such as, for example, by reading a QR code, detecting a color, and/or reading text, numerals, and/or symbols disposed on the surface of cartridge 116 or on the surface of a container holding cartridge 116. Alternatively, or in addition, cartridge sensor 108 may comprise a RFID or NFC sensor that reads a RFID and/or NFC tag integrated with, coupled with, or associated with cartridge 116 (or with a container holding cartridge 116). In some embodiments, cartridge sensor 108 may also comprise a tactile sensor (e.g., a switch) that is triggered or actuated by a portion of cartridge 116 when cartridge 116 is placed within medication storage chamber 118.

The one or more additional sensors 117 are optional, but may include, in some embodiments, sensors configured to record, sense, and/or measure an ambient temperature surrounding the hub 100, an ambient humidity surrounding the hub 100, an amount of light incident upon hub 100, and/or an internal temperature of hub 100. In some embodiments, sensor(s) 117 may detect whether hub 100 is plugged into a power source or not. In embodiments where hub 100 comprises a battery (not shown) that provides power and allows hub 100 to operate even when not plugged into a power source, sensor(s) 117 may detect a level of charge remaining within said battery. In some embodiments, sensor(s) 117 may also detect a status of movable door 114, e.g., whether the door is open, shut, locked, and/or unlocked.

Communication device 110 may be a wireless communication device that enables wireless communication between hub 100 and reusable drug-delivery device 150, and/or between hub 100 and a mobile device 180. For example, communication device 110 may establish a communication link 195 with mobile device 180. Examples of suitable wireless communication devices include chip packages, circuits, and/or antenna for sending and/or receiving Bluetooth, WiFi, RFID, or NFC signals. Alternatively, or in addition, communication device 110 may be a wired communication device that enables wired communication with the aforementioned devices, e.g., through a Universal Serial Bus (USB) connection. Alternatively, or in addition, communication device 110 may comprise a long-rang cellular communication interface that establishes a communication link with a server 170 via network 198 and communication links 196 and 199. The server 170 may be located remote from hub 100 and/or mobile device 180, e.g., in another building, in another city, or even in another country or continent. Network 198 may comprise any cellular or data network adapted to relay information between hub 100, mobile device 180, and/or server 170, potentially via one or more intermediate nodes or switches. Examples of suitable networks 170 include a cellular network, a metropolitan area network (MAN), a wide area network (WAN), and the Internet.

Mobile device 180 may comprise a user's mobile or processing device, such as the user's smartphone, smartwatch, tablet, laptop, pager, and/or desktop. The mobile device 180 may comprise a processor 182 capable of executing programmable code through control logic 184. Mobile device 180 may also comprise memory 186 that may store control logic 184. The memory 186 storing the software/firmware code is any suitable computer readable medium that is accessible by the at least one processor. The memory may be a single storage device or multiple storage devices, may be located internally or externally to the at least one processor, and may include both volatile and non-volatile media. Exemplary memory includes random-access memory (RAM), read-only memory (ROM), electrically erasable programmable ROM (EEPROM), flash memory, a magnetic storage device, optical disk storage, or any other suitable medium which is configured to store data and which is accessible by the at least one processor. The mobile device 180 may also include a display 188 for displaying information for a user and/or receiving input from the user (in the case of a touch-sensitive display) and a communication device 190 for communicating with external devices (e.g., with hub 100 via communication link 195). In some embodiments, communication device 190 may also comprise a cellular and/or data interface for establishing data communication with server 170 via network 198 and communication links 197 and 199.

Server 170 illustratively includes any computing device configured to receive and process data from mobile device 180 and/or hub 100 via network 198. Such data received by server 170 may comprise any data based on or derived from raw data gathered, measured, and/or recorded by hub 100 and/or drug-delivery device 150. For example, data received by server 170 may include the raw data gathered, measured, and/or recorded by hub 100 and/or drug-delivery device 150. Alternatively, or in addition, the data received by server 170 may include any data generated or derived by hub 100 and/or mobile device as a result of processing such raw data. Examples of data that may be sent to server 180 from hub 100 and/or mobile device 180 may include, for example, time and amount of doses delivered by drug-delivery device 150 to a user, an amount of drug remaining within hub 100, an expiration date of any drugs remaining within hub 100, a temperature excursion, power outage, low battery condition, or other fault condition sensed by hub 100, an indication that the hub 100 has exhausted (or is close to exhausting) its store of drug, a request from a user for more drug to replenish said store of drug within hub 100, an indication of how well the user is adhering to a predetermined medication regimen based on the user's dosage history, and the like. Such information may be transmitted directly to server 170 from hub 100 via network 198 (and all intervening communication links). Alternatively, or in addition, such information may be transmitted to server 170 from hub 100 via mobile device 180, as well as network 198 and all intervening communication links.

Server 170 may process and/or store said information, and optionally, send responses, notifications, or instructions to either mobile device 180, hub 100, or other devices not shown in FIG. 1. Server 170 includes processing circuit 172, memory 174, and communications device 176. Processing circuit 172 may include any of the possible types of processors and/or processing circuits previously described. Processing circuit 172 may execute software and/or firmware stored in memory 174 of server 170. The software/firmware code contains instructions that, when executed by processing circuit 172, cause the server 172 to perform the functions described herein. Memory 174 may also be configured to store information regarding one or more users of hub 100 and/or drug-delivery device 150, such as biographical information and/or medical information (e.g., medication dosing records, medical history, and the like). Information received from or sent to either hub 100 and/or mobile device 180 may also be stored in memory 174. Memory 174 may include any of the possible types of memory previously described. Communication device 176 allows server 170 to communicate with mobile device 180 and/or hub 100 via network 198 and communication links 196, 197, and/or 199. Without limiting the generality of the foregoing, in some embodiments, server 170 may comprise, be communicatively coupled to, or implement all or part of an Electronic Health Record (EHR) system maintained by a healthcare provider (HCP) or a healthcare organization or institution, such as a hospital system or physician's office. In such embodiments, the server 170 may serve to keep records (e.g., time and/or amount of medication dosage) pertaining to a user's usage of hub 100 and/or drug-delivery device 150 to assist the HCP and/or healthcare organization to provide care for said user. In other embodiments, server 170 may be maintained by a pharmaceutical company, a manufacturer of medical devices (e.g., of hub 100 and/or drug-delivery device 150), a health insurance payer, and/or health outcome researchers conducting a clinical trial or study.

Returning to hub 100, in some embodiments, controller 104 includes at least one processor that executes software and/or firmware stored in a memory of hub 100. The software/firmware code contains instructions that, when executed by the at least one processor, causes hub 100 to perform the functions described herein. The at least one processor illustratively includes control logic operative to, for example: operate the temperature-changing device 112 to cool the cartridge 116 when it is being held in storage, operate the temperature-changing device 112 to heat the cartridge 116 when it receives user input indicating the cartridge 116 is about to be used, operate the user-interface to convey information regarding the status of hub 100 and/or drug-delivery device 150 (e.g., a battery charge of delivery device 150; type(s), use(s), recommended dose(s), and/or expiration date(s) of medication stored within hub 100; an internal temperature of one or more medical storage chambers 118; whether movable door 114 is open, shut, or locked; whether hub 100 is properly paired with a user's mobile device or drug-delivery device, and the like), and/or operate the user-interface to receive user-input (e.g., input instructing hub 100 to turn on or off, pair with delivery device 150 and/or a user's mobile device, prepare one of the cartridges 116 for a dose, to unlock, lock, open, or close movable door 114, and the like). The memory storing the software/firmware code is any suitable computer readable medium that is accessible by the at least one processor, including any of the types of memory previously described in relation to memory 186 of mobile device 180. In some embodiments, controller 104 may include (or consist essentially of) an application-specific integrated circuit (ASIC) configured to perform the above-mentioned functions.

Reusable drug-delivery device 150 may comprise a housing 151 that houses or supports the various components of device 150. Device 150 comprises a coupling mechanism 168 that, when operated, releasably couples with medication cartridge 116. Device 150 also comprises a drive mechanism 154 that, when medication cartridge 116 is coupled with coupling mechanism 168, delivers medication from medication cartridge 116 to a patient. In one embodiment, medication cartridge 116 may comprise a syringe having a barrel that holds the medication, a needle, and a movable plunger that, when depressed, forces medication out of the needle. In such embodiments, drive mechanism 154 may comprise a movable drive member that, when drive mechanism 154 is operated, pushes against the movable plunger to force medication out of cartridge 116. In other embodiments, medication cartridge may not include a movable plunger or a needle. In such embodiments, drive mechanism 154 may comprise a pump to draw the medication out of medication cartridge 116 and to deliver the medication to the patient, e.g., through a needle, a jet injection, orally, and/or intravenously. Drive mechanism 154 may be coupled with a trigger button 152 that, when actuated by a user, operates the drive mechanism 154.

Device 150 may further include an electronics package 156, e.g., one or more printed circuit boards (PCBs) or electronic circuits. Although shown as a single discrete package in FIG. 1, package 156 may be dispersed through multiple locations throughout drug-delivery device 150 in some embodiments. Package 156 may comprise a controller 160, one or more device sensors 162, a communication device 164, and/or a user-interface 166. Package 156 may also comprise the aforementioned rechargeable power source 158 configured to provide power to some or all of the other aforementioned components of package 156. When charging contact 170 on device 150 is brought into contact or close proximity with charging station 102, charging station 102 may replenish an electrical charge within rechargeable power source 158.

Device sensors 162 may comprise one or more sensors configured to sense, record, measure, or detect a status of reusable drug-delivery device 150. For example, device sensors 162 may be configured to sense whether device 150 is powered on or off, whether device 150 has been coupled to a medication cartridge 116, whether device 150 is turned on or off, whether device 150 is locked or unlocked for dosing, whether device 150 has been triggered to dose, whether device 150 has started its dose, whether device 150 has completed its dose, an amount of dose programmed into and/or delivered by device 150, a position or movement of a movable component within device 150 (e.g., trigger button 152, a movable drive member within drive mechanism 154, and/or a movable plunger within medication cartridge 116), a current electrical charge present within rechargeable power source 158, a charge capacity of rechargeable power source 158, whether or not power source 158 is currently being charged, proximity of device 150 to hub 100, proximity and/or contact between device 150 and human tissue, such as a user's skin, an acceleration and/or orientation of device 150, a current external or internal temperature of device 150 or of a medication within medication cartridge 116, a type of medication stored within medication cartridge 116, and/or a current geographic location of device 150. Sensors 162 may comprise one or more optical sensors, electrical sensors, magnetic sensors, accelerometers, mechanical switches, wireless antenna, and/or GPS sensors.

Communication device 164 may take the form of any of the devices described above for communication device 110. Similarly, user-interface 166 may take the form of any of the devices described above for user-interface 106. Controller 160 may be configured to receive data from and/or control the operation of any of device sensors 162, communication device 164, and/or user-interface 166. Controller 160 may take the form of any of the devices described above for controller 104. In some embodiments, controller 160 may be configured to report any of the aforementioned types of data sensed by sensors 162, or any data derived from any of the aforementioned types of data, to hub 100 via communication device 164. As previously described, hub 100, in turn, may forward such data to server 170 via network 198 (and/or optionally via mobile device 180), or may send to server 170 data derived from or generated based on the aforementioned types of data sensed by sensors 162.

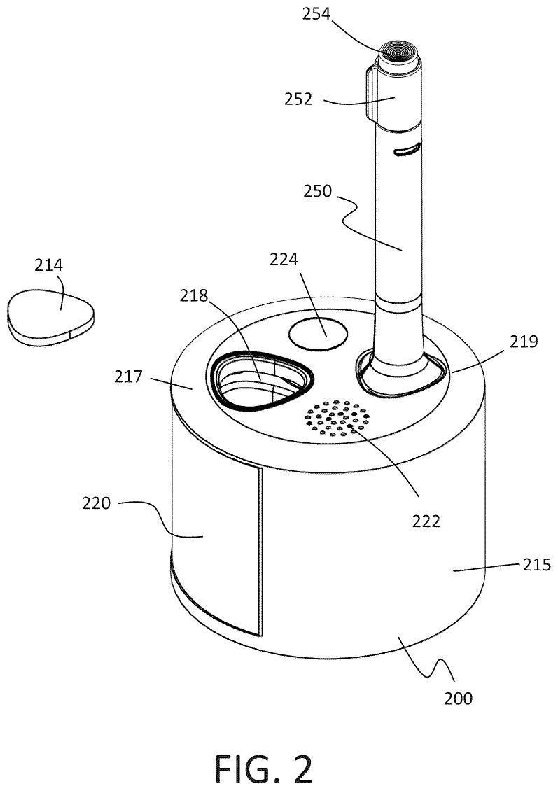

FIG. 2 provides a perspective view of a first embodiment of a hub for holding a reusable drug-delivery device. Hub 200 and drug-delivery device 250 may be configured similarly to hub 100 and drug-delivery device 150, respectively. In this first embodiment, drug-delivery device 250 takes 1 the form of an autoinjector having a proximal end (pointing towards the bottom of FIG. 2) and a distal end (pointing towards the top of FIG. 2). To use the drug-delivery device 250, a user presses the proximal end of drug-delivery device 250 against the patient's skin, then unlocks the device by rotating a locking element 252. After the device is unlocked, the user triggers the device by pressing down on trigger button 254. Once triggered, drug-delivery device 250 activates a drive mechanism to deliver an injection to the patient.

In this embodiment, hub 200 is substantially shaped as a cylinder having a curved side wall 215 and a top surface 217. Top surface 217 defines a port 218 that is covered by a movable (or removable) door 214. Although door 214 is shown completely detached from top surface 217 in FIG. 2, door 214 may also be configured to hinge or slide open or closed in some embodiments. Top surface 217 also includes a charging station 219 which, in this embodiment, takes the form of a shallow receptable configured to receive the proximal end of drug-delivery device 250. When the proximal end of drug-delivery device 250 is received within the shallow receptable of charging station 219, station 219 may replenish an electrical charge within a rechargeable power source mounted within device 250. Both port 218 and the walls of the shallow receptable of charging station 219 may be shaped (such as circular, triangular, rectangular, etc.) to snugly receive the outer shape of the proximal end of drug-delivery device 250.

Top surface 217 of hub 200 further includes a button 224 and a speaker 222. Hub 200 may be configured to report a status of hub 200 and/or device 250 when a user presses button 224. For example, the hub 200 may be configured to report on a charge status of device 250, on a temperature of one or more drugs or medication cartridges stored within hub 200, on an expiration date of the one or more drug or medication cartridges, a type of one or more medications stored within hub 200, a notice to replace one or more medication cartridges, and/or one or more detected fault conditions. The status report may be delivered audibly through speaker 222, or through a wireless signal sent to the user's mobile device. Although this embodiment incorporates a speaker, other means for providing an indication or status report to a user are also contemplated, including LEDs, displays, and/or haptic indicators.

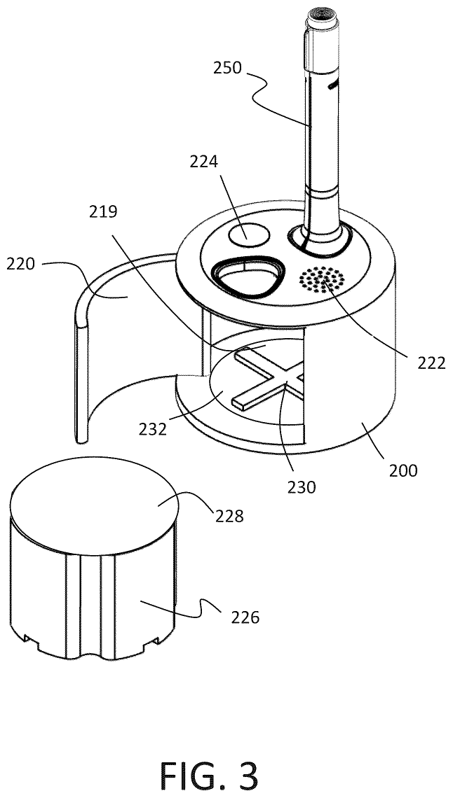

In this embodiment, the curved side wall 215 of hub 200 includes a hinged door 220. FIG. 3 provides a perspective view of the interior of hub 200 when door 220 is opened. The interior of hub 200 may define a hollow space 219 that is configured to accommodate a medication bin 226. The floor of the hub 200 may include a rotating turntable 232 that rotates about a central axis 239 relative to the hub. Arranged on top of the rotating turntable 232 can be protruding guides 230 (in this embodiment, shaped as a cross) extending into the hollow space 219. As described in further detail below, guides 230 are configured to fit into corresponding grooves on the underside of medication bin 226.

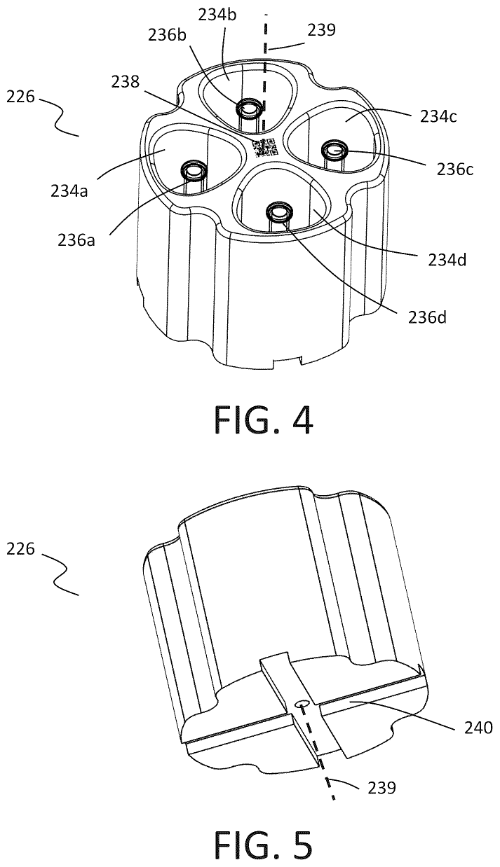

Medication bin 226 may also be substantially shaped as a cylinder. The top surface of bin 226 may be sealed by a removable sterile barrier 228 that isolates and maintains the sterility of compartments within bin 226. FIG. 4 provides a top perspective view of medication bin 226 when sterile barrier 228 is removed. Underneath sterile barrier 228, bin 226 includes any number of storage chambers (shown as four separate medication storage chambers 234a, b, c, d) (collectively or individually referred to herein as medication storage chamber 234, as appropriate). Each medication storage chamber may be provided with the aforementioned temperature-changing device 112 to either cool or heat the interior of the chamber. Each chamber may also be configured to hold a medication cartridge 236a, b, c, d (collectively or individually referred to herein as medication cartridge 236, as appropriate). Each cartridge 236a, b, c, d may be configured similarly to the aforementioned cartridge 116. In some embodiments, bin 226 may further comprise a QR code 238 disposed on a top surface thereof. When scanned by a suitable optical sensor, e.g., on a mobile device or installed on or within hub 200, the QR code may provide information regarding the type(s) of medication stored within bin 226, the expiration date(s) of such medication, the manufacturing facility or lot that produced such medication, and/or the ideal storage or delivery parameters for such medication (e.g., the ideal storage or delivery temperature).

FIG. 5 provides a bottom perspective view of medication bin 226. As can be seen, the underside of medication bin 226 includes recessed guides 240 defined therein which, in this embodiment, take the shape of a cross. When bin 226 is positioned on top of the rotating turntable 232 within hub 200, the protruding guides 230 fit into the recessed guides 240 and inhibit rotational movement of bin 226 relative to turntable 232. When bin 226 is so positioned, one of the medication storage chambers 234a, b, c, d will be aligned with the port 218. By rotating turntable 232, bin 226 may be rotated about its central axis 239 to position another of the medication storage chambers 234 in alignment with port 218.

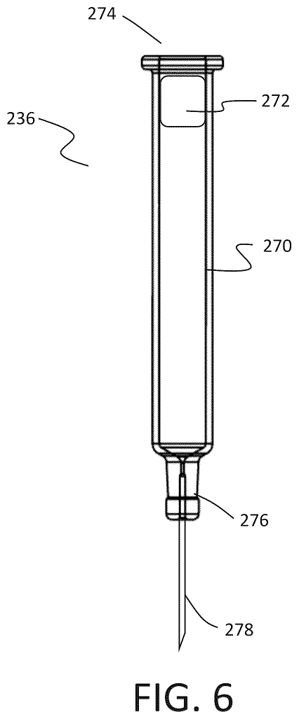

FIG. 6 provides a profile view of an exemplary medication cartridge 236 stored within a medication storage chamber 234. Cartridge 236 may comprise a barrel 270 having a proximal end 276 and a distal end 274. A distal end of barrel 270 may be sealed with a movable plunger 272, while a proximal end of barrel 270 may be capped with a hollow cannula 278 configured to penetrate a person's skin. Medication may be stored within barrel 270 between the movable plunger 272 and the cannula 278. When the movable plunger is pushed toward the cannula 278 by, for example, a drive mechanism within drug-delivery device 250, the movement of the plunger may force medication out of cannula 278.

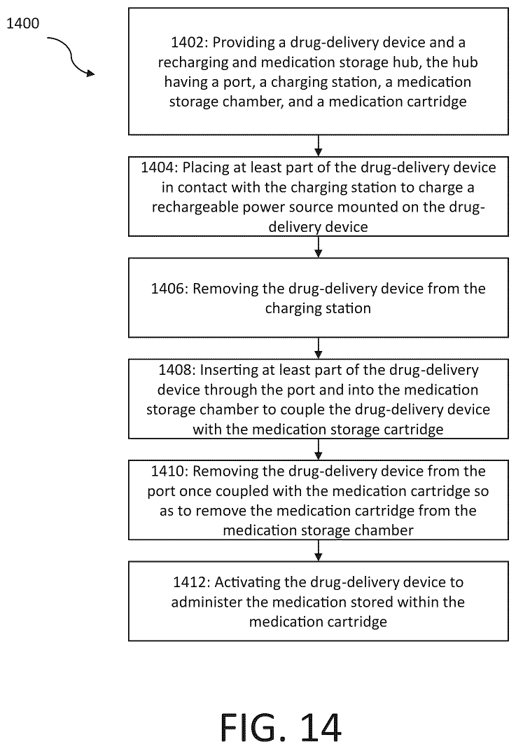

FIG. 14 depicts a flow-chart illustrating an exemplary process 1400 for using drug-delivery device 250 and hub 200. At step 1402, a drug-delivery device (e.g., re-usable drug-delivery device 250) and a recharging and medication storage hub (e.g., hub 200) are provided. As previously discussed, the hub has a port (e.g., port 218), a charging station (e.g., station 219), a medication storage chamber (e.g., chamber 234) in alignment with the port, and a medication cartridge (e.g., single-use medication cartridge 236) disposed within the medication storage chamber.

At step 1402, at least part of the drug-delivery device is placed in contact with the charging station to charge a rechargeable power source mounted on the drug-delivery device.

At step 1404, a user may remove the drug-delivery device from the charging station, e.g., by picking up drug-delivery device 250 from charging station 219.

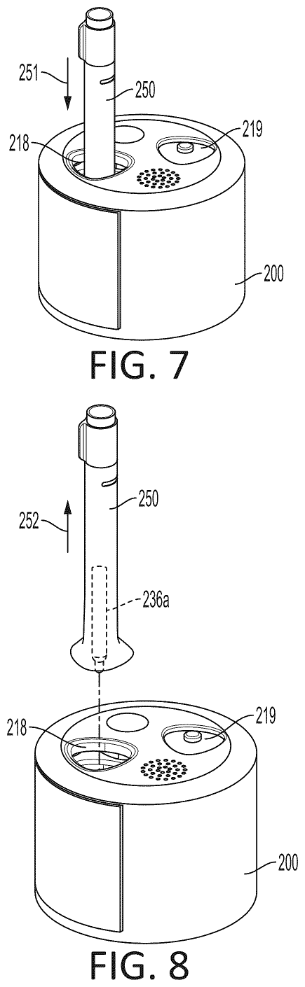

At step 1408, the user may insert at least part of the drug-delivery device through the port and into the medication storage chamber to couple the drug-delivery device with the medication cartridge stored within the medication storage chamber. For example, as shown in FIG. 7, the user may insert the proximal end of device 250 through port 218 and into a medication storage chamber 234a, i.e., by pushing device 250 downward in the direction of arrow 251. The downward movement of device 250 causes a coupling mechanism within device 250 (e.g., a coupling mechanism 168) to releasably couple with a medication cartridge 236 (e.g., cartridge 236a). For example, the coupling mechanism 168 may comprise flexible members (e.g., fins, ridges, gates) that bend when pressed downward by device 250, and that snap back into place around a flange, ridge, depression, or other feature within device 250 to releasably couple the medication cartridge 236 to device 250. As another example, coupling mechanism 168 may comprise a magnet that couples magnetically with a magnetic or metallic ring, fin, or other structural element mounted on cartridge f236 to releasably couple the medication cartridge 236 to the inside of device 250.

At step 1410, after the medication cartridge is coupled with the drug-delivery device, the user may remove the drug-delivery device 250 from the port 218 (e.g., by pulling in the direction of arrow 252; see FIG. 8) so as to remove the medication cartridge 236a from the medication storage chamber 234a. Device 250 is now ready to deliver the medication within the coupled medication cartridge 236a.

At step 1412, the user may activate the device 250 by placing device 250 in contact with the patient's body and actuating an activation button to administer the medication within the medication cartridge.

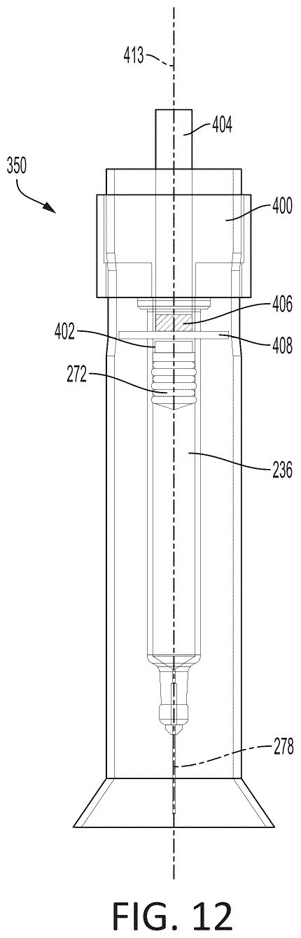

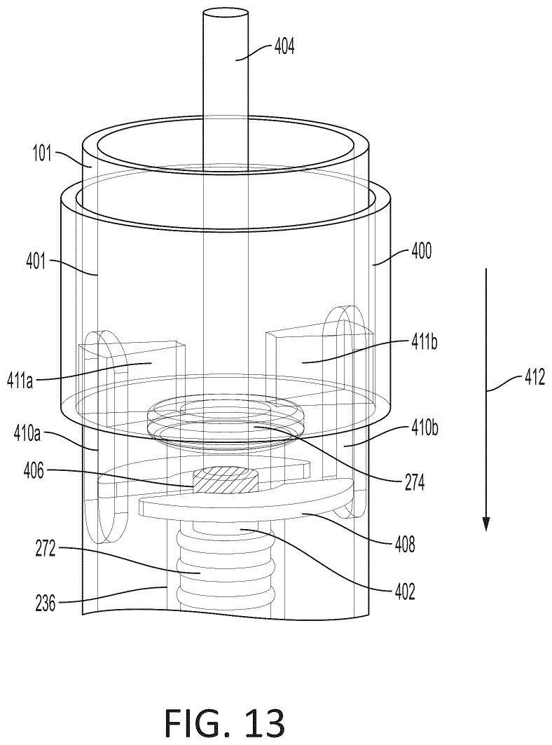

When the medication within cartridge 236a has been exhausted, the user may once again push the proximal end of device 250 through port 218 and into the empty medication storage chamber 234a. The coupling mechanism 168 releases the cartridge 236a into the storage chamber 234a. For example, the user may slide a release sleeve to release the cartridge, as discussed in further detail below in relation to FIGS. 12-13. Alternatively, or in addition, the user may press a button or actuate a switch on the body of device 250 to release the snap-fit member or magnetic member that is holding onto cartridge 236a. The user can then replace the reusable drug-delivery device 250 back onto the charging station 219 to recharge the device.

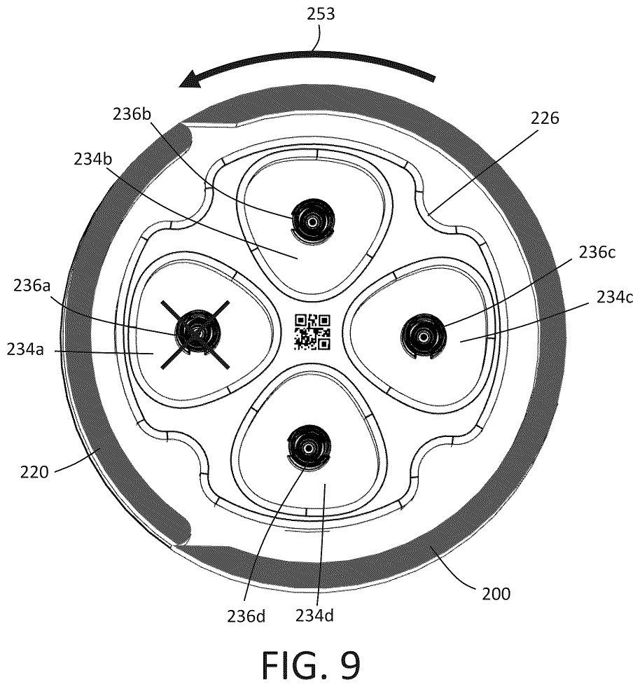

After the aforementioned operations, the user may rotate the turntable 232 relative to the hub in any direction such as in the direction of arrow 253 (see FIG. 9). This rotates the bin 226 about its central axis 239, which moves the exhausted cartridge 236a out of alignment with port 218 and moves the next cartridge 236b into alignment with port 218. The new cartridge 236b is now ready to be loaded into drug-delivery device 250 for another dose. In this manner, all cartridges 236a, b, c, d may be loaded, exhausted, and replaced into medication storage chambers 234a, b, c, d. When all four cartridges 236a, b, c, d have been exhausted, the entire bin 226 may be removed from hub 200. The entire bin 226 may then be discarded and a new bin holding fresh cartridges may be inserted into hub 200. In this way, the bin 226 may serve both as a container for new cartridges 236, and also as a disposable sharps container for used cartridges 236.

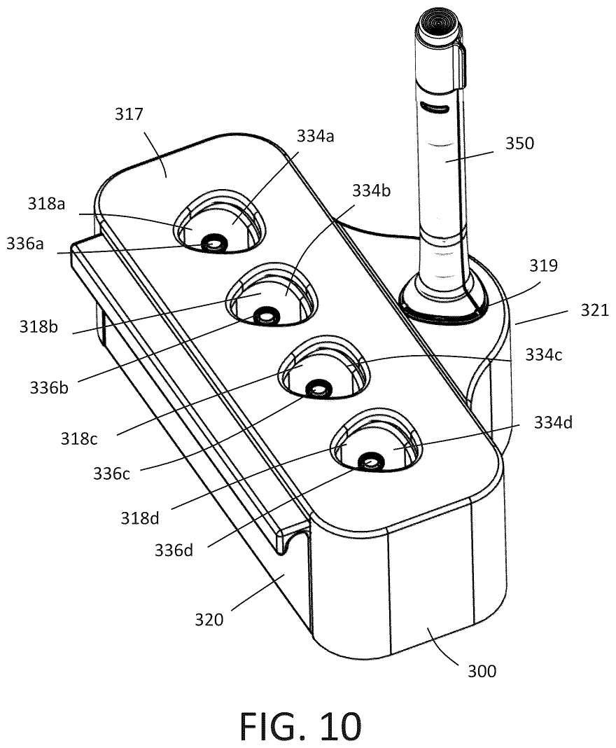

FIG. 10 provides a perspective view of a second embodiment of a hub for holding a reusable drug-delivery device. Hub 300 and drug-delivery device 350 may be configured similarly to hub 100 and drug-delivery device 150, respectively. In this second embodiment, drug-delivery device 350 may also take the form of an autoinjector having a proximal end (pointing towards the bottom of FIG. 10) and a distal end (pointing towards the top of FIG. 10). When the proximal end of drug-delivery device 350 is pressed against a patient's skin, drug-delivery device 350 may be triggered to deliver an injection to the patient.

In this embodiment, hub 300 is shaped as a substantially rectangular block having a top surface 317. Top surface 317 defines a plurality of ports (four in this embodiment, although embodiments with more or fewer ports are also contemplated) 318a, b, c, d (collectively or individually referred to herein as ports 318, as appropriate). Each port may be covered by a movable or removable door (not shown). Each port provides access to a separate medication storage chamber 334a, b, c, d within a removable bin 320. Similar to the port 218 and receptable of charging station 219 in hub 200, each port 318 a, b, c, d may be shaped and/or configured to snugly receive the shape of the proximal end of drug-delivery device 350. Hub 300 also includes a rounded lateral protrusion 321 that extends from one side of the rectangular block. The top surface of the rounded protrusion 321 includes a charging station 319 on the top surface 317 of hub 300, which, in this embodiment, can have the shape of the proximal end of the device.

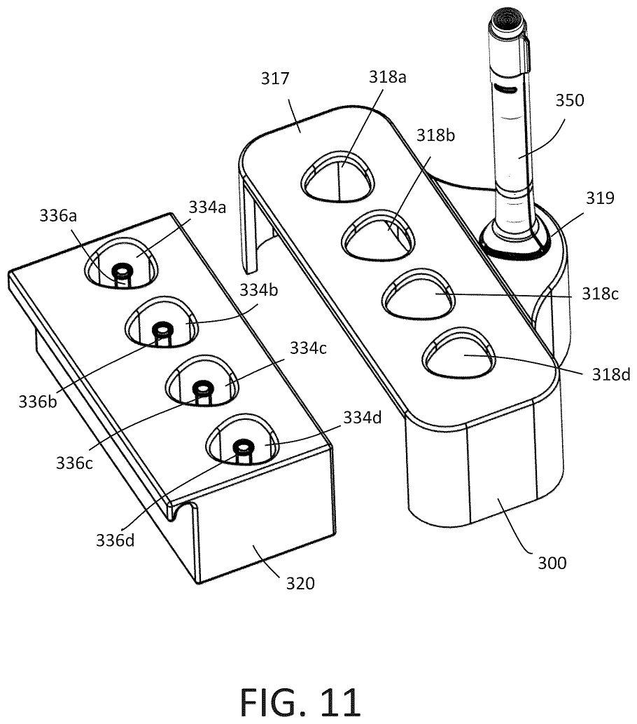

Hub 300 further includes a removable bin 320. FIG. 11 shows a perspective view of hub 300 in which the bin 320 has been removed. As shown, bin 320 may also take the form of a smaller rectangular block that is configured to slide under the top surface 317 of hub 300. Bin 320 defines multiple (four in this embodiment) medication storage chambers 334a, b, c, d (collectively or individually referred to as medication storage chambers 334, as appropriate). Each storage chamber 334 a, b, c, d may be provided with a temperature-changing device discussed above to either cool or heat the interior of the chamber. Each chamber may also be configured to hold a medication cartridge 336a, b, c, d (collectively or individually referred to as medication cartridge 336, as appropriate). Each cartridge 336 a, b, c, d may be configured similarly to the aforementioned cartridge 116 and/or cartridge 236. Although not shown, bin 320 may also include a QR code similar to QR code 238 discussed above.

FIG. 12 provides a profile view of the drug-delivery device 350 with a medication cartridge 236 coupled via a coupling mechanism. In one embodiment, the coupling mechanism comprises a magnetic coupling mechanism. For example, the coupling mechanism may comprise a first magnet 402 attached to a distal surface of the movable plunger 272 of the medication cartridge 236, a second magnet 406 attached to a proximal surface of a plunger rod 404 of the re-usable drug-delivery device 350, and a release sleeve 400 (described in further detail below in FIG. 13). When the medication cartridge is coupled to the coupling mechanism, first magnet 402 and second magnet 406 may be disposed along a central longitudinal axis 413 of the drug-delivery device 350. When the proximal end of re-usable drug-delivery device 350 is inserted into the medication storage chamber 234, the distal end of the medication cartridge 236 containing the first magnet 402 moves distally inside the re-usable drug-delivery device 350 until the attractive force of the first magnet 402 and second magnet 406 is strong enough to hold the medication cartridge 236 in place inside re-usable drug-delivery device 350. When the user proceeds to deliver medication from the re-usable drug-delivery device 350 after coupling with the medication cartridge 236, the user triggers the drive mechanism 154 which proximally moves plunger rod 404 and movable plunger 272 towards the cannula 278, forcing medication out of cannula 278.

FIG. 13 provides a detailed view of the proximal end of the plunger rod 404 within re-usable drug-delivery device 350, the distal end of medication cartridge 236, and the release sleeve 400. Release sleeve 400 comprises a hollow cylindrical body 401 that wraps around an outer surface of drug-delivery device 350. Release sleeve 400 further comprises two release tabs 411a and 411b (individually or collectively referred to as tabs 411, as appropriate) which project radially inward from the inner surface of cylindrical body 401 into an interior volume of body 401. Tabs 411a and 411b are received within axially-extending grooves 410a and 410b (individually or collectively referred to as grooves 410, as appropriate) which are defined within housing 101 of reusable drug-delivery device 350. To de-couple the medication cartridge 236 from re-usable drug-delivery device 350, the user may grasp cylindrical body 401 of release sleeve 400 and push release sleeve 400 in the direction of arrow 412 (i.e., in the proximal direction). As release sleeve 400 moves proximally, tabs 411 move proximally within grooves 410 and exert a proximal force on distal end 274 of the medication cartridge 236. This proximal force overcomes the magnetic force holding first magnet 402 and second magnet 406 together, thus releasing the medication cartridge 236. To assist with ensuring a stable coupling of medication cartridge 236 within medication delivery device 350 and with ensuring alignment of magnets 402 and 406, a guide member 408 may be used to keep the medication cartridge 236 from excessive movement during utilization of the re-usable drug-delivery device 350. As shown in FIG. 13, guide member 408 may take the form of two arcuate shaped tabs that are sized to fit tightly within an interior circumference of medication delivery device 350. However, guide members 408 may take other forms, such as a flange, an O-ring, or a plurality of fins that are received within guidance slots defined on the interior surface of medication delivery device 350. If necessary, more than one guide member 408 may be used to improve stability and reduce unwanted movement of the medication cartridge 236 while coupled with the re-usable drug-delivery device 350. Guide member 408 may form part of the medication cartridge 236 or of the drug-delivery device housing 101.

In one example, an exemplary method for using a recharging and medication storage hub is provided. The hub consists of a port, a charging station, a medication storage chamber in alignment within the port, and a single-use medication cartridge storing a medication disposed within the medication storage chamber. A user may recharge a rechargeable power source mounted on the re-usable drug-delivery device by placing at least part of the re-usable drug-delivery device in contact with the charging station. When the user is ready to administer a medication, the user may remove the drug-delivery device from the charging station and insert at least part of the drug-delivery device through the port and into the medication storage chamber to couple the medication cartridge with the drug-delivery device and then remove the drug-delivery device from the medication storage chamber and administer the medication. The hub may include a product identification system such as for example a Quick Response (QR) code disposed on the hub wherein the user can scan the code to receive information regarding the medication. Alternatively, the hub may also include a sensor (e.g., cartridge sensor 108) that is configured to scan the QR code. The hub may also issue instructions or provide indications to the user via at least one of an audio speaker disposed on the hub, one or more visual indicators disposed on the hub, and a wireless transmission to a mobile device, wherein a visual indicator may include any one of or any combination of one or more LEDs, light rings, light panels, and displays. A smartphone may be connected to the hub where the user can issue commands to instruct the hub to open, close, lock, or unlock a door to the port or instruct a temperature-changing device to keep the interior temperature of the storage chamber at a storage temperature and then subsequently instruct the hub to increase the temperature inside the storage chamber to an administration temperature.

In one embodiment, the method may further comprise depositing a used medication cartridge back into the medication storage chamber from which the medication cartridge was removed. To do so, the use may insert the drug-delivery device through the port into the medication storage chamber from which the medication cartridge was obtained and decouple the medication cartridge from the drug-delivery device. In some embodiments, the hub may consist of a plurality of single-use medication cartridges, where the medication storage chamber is one of a plurality of medication storage chambers, each medication storage chamber being configured to hold one single-use medication cartridge of the plurality of medication cartridges. In some embodiments, the plurality of medication storage chambers may be defined within a movable bin. In such embodiments, the method may further comprise moving the bin to position another of the plurality of storage chambers in alignment with the port after the drug-delivery device has removed and/or used one of the single-use medication cartridges.

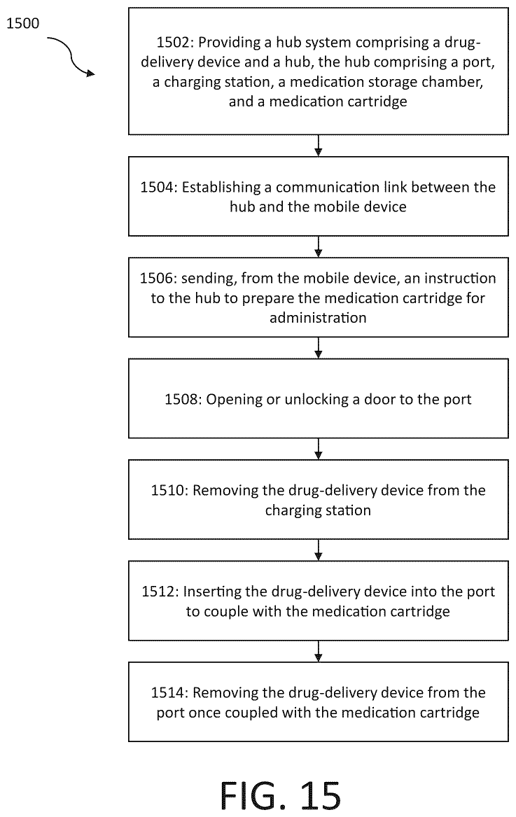

FIG. 15 is a flow chart depicting an exemplary process 1500 for using a recharging and medication storage hub with a mobile device. The process begins with step 1502, in which a hub system is provided. The hub system comprises a drug-delivery device (e.g., device 150, 250, 350) having a rechargeable power source and a hub (e.g., hub 100, 200, 300). The hub may comprise a port, a charging station, a medication storage chamber, and a medication cartridge. The port may have a door that is configured to move between an open and closed position or switch between a locked and unlocked configuration. The charging station is configured to charge (either directly or inductively) the re-chargeable power source on the re-usable drug delivery device when at least a portion of the drug-delivery device is placed in contact with the charging station. The medication storage chamber is designed to be aligned with the port. The medication cartridge is disposed within the medication storage chamber.

At step 1504, process 1500 establishes a communication link between the hub and the mobile device (e.g., device 180). As previously discussed, the mobile device may be any of, but not limited to, the following devices: a smartphone, a laptop, a pager, a smartwatch, a tablet, a desktop, or any device which can receive a transmission and subsequently send a responding transmission. The communication link may wireless or wired, and may be achieved by any of, but not limited to, the following communication protocols or technologies: Bluetooth, RFID, NFC, fiber-optic communication, Universal Serial Bus (USB) communication, wireless networks such as Wi-Fi, cellular networks, or infrared (IR).

At step 1506, once the communication link is established, the mobile device may send an instruction to the hub to prepare the medication for administration. The instruction may be issued from the mobile device based on user input at the mobile device and/or based on a pre-programmed schedule for administration of the medication.

At step 1508, in response to the received instruction to prepare the medication for administration, the hub may then open or unlock the door to the port to allow access to the medication cartridge. The hub may, but is not required to, issue one or more indications to the user that the medication is ready for administration. The indications may be issued via any one of or any combination of one or more audible sounds generated by at least one audio speaker disposed on the hub, one or more visual indicators, and a wireless transmission to a mobile device. Visual indicators may further comprise any one of or any combination of one or more LEDs, light rings, light panels, and/or displays. The indications may be issued before or after the port is unlocked or opened at step 1508.

At step 1510, the user may then remove the drug-delivery device from the charging station. Once removed from the charging station, the user may then, at step 1512, insert the drug-delivery device into the port to couple the drug-delivery device with the medication cartridge. After coupling of the drug-delivery device and the medication cartridge, at step 1514, the user may then remove the drug-delivery device so as to remove the medication cartridge from the medication storage chamber. Once removed from the port, the user may activate the drug-delivery device to administer the medication. The process 1500 may further include disposing of the medication cartridge by inserting the drug-delivery device into the port and decoupling the used medication cartridge from the drug-delivery device to deposit the used cartridge in the medication storage chamber. For example, the user may move the release sleeve 400 (depicted in FIGS. 12-13) in the proximal direction to decouple the medication cartridge from the drug-delivery device.

In some embodiments, the hub may further comprise a temperature-changing device configured to control an interior temperature of the medication storage chamber. The temperature-changing device may regulate the interior temperature of the medication storage chamber, e.g., by cooling the interior temperature of the medication storage chamber to a storage temperature (e.g., 30-34 degrees Fahrenheit). In such embodiments, in response to the instruction to prepare the medication for administration (step 1506), the hub may raise the interior temperature of the medication storage chamber to an administration temperature (e.g., room temperature) using the temperature-changing device. Optionally, once the interior temperature of the storage chamber has reached the administration temperature, the hub may issue one or more indications using the previously mentioned indication method(s) to inform the user that the medication cartridges are ready for administration, e.g., using speaker 222 or user-interface 106.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

Various aspects are described in this disclosure, which include, but are not limited to, the following aspects:
1. A recharging and medication storage hub for a drug-delivery device, the hub comprising: a housing defining a port sized to receive at least part of the drug-delivery device; a charging station disposed on the housing configured to charge a rechargeable power source mounted on the drug-delivery device when at least part of the drug-delivery device is positioned in proximity with the charging station; and a medication storage chamber configured to align with the port, the medication storage chamber configured to store a medication cartridge; wherein when at least part of the drug-delivery device is inserted through the port and into the medication storage chamber and then withdrawn from the medication storage chamber, the medication storage chamber is configured to allow the drug-delivery device to withdraw the medication cartridge from the medication storage chamber.
2. The hub of aspect 1, wherein the medication cartridge comprises a syringe having a barrel storing a medication, a needle, and a movable plunger.
3. The hub of any of aspects 1-2, further comprising the medication cartridge, the medication cartridge storing a medication.
4. The hub of any of aspects 1-3, wherein: the drug-delivery device comprises a proximal end and a distal end; and the port is configured to receive at least the proximal end of the drug-delivery device.
5. The hub of any of aspects 1-4, further comprising a cooling device configured to lower an interior temperature of the medication storage chamber.
6. The hub of any of aspects 1-4, further comprising a heating device configured to raise an interior temperature of the medication storage chamber.
7. The hub of any of aspects 1-4, further comprising a temperature-changing device configured to both lower and raise an interior temperature of the medication storage chamber.
8. The hub of any of aspects 1-7, wherein: the medication cartridge is a first medication cartridge of a plurality of medication cartridges, the medication storage chamber is a first medication storage chamber of a plurality of medication storage chambers defined within a bin disposed at least partially within the housing; and each medication storage chamber of the plurality of medication storage chambers is configured to store one medication cartridge of the plurality of medication cartridges.
9. The hub of aspect 8, wherein the bin is movable so as to position only one of the plurality of medication storage chambers in alignment with the port at a time.
10. The hub of aspect 9, wherein the bin is configured to rotate so as to position only one of the plurality of medication storage chambers in alignment with the port at a time.
11. The hub of any of aspects 1-7, wherein: the port is a first port of a plurality of ports defined in the housing, each port sized to receive at least part of the drug-delivery device; the medication cartridge is a first medication cartridge of a plurality of medication cartridges; and the medication storage chamber is a first medication storage chamber of a plurality of medication storage chambers, each medication storage chamber disposed in alignment with a separate port of the plurality of ports, and each medication storage chamber being configured to store one medication cartridge of the plurality of medication cartridges.
12. The hub of any of aspects 1-11, wherein the medication storage chamber is configured to store the medication cartridge after the medication cartridge is used and de-coupled with the drug-delivery device.
13. The hub of any of aspects 1-12, wherein the hub further comprises: a communication device; and a controller configured to: receive, via the communication device, data recorded by sensors mounted on the drug-delivery device, and send, via the communication device, information based on such recorded data to a remote server.
14. The hub of any of claims 1-12, wherein the hub further comprises: one or more sensors configured to record data indicative of a status of the hub; a communication device; and a controller configured to send, via the communication device, data based on said recorded data to a remote server.
15. A system for delivering and storing medication, the system comprising the recharging and medication storage hub and the drug-delivery device of any of aspects 1-14.
16. The system of aspect 15, wherein the drug-delivery device comprises a coupling mechanism configured to releasably couple with the medication cartridge.
17. The system of aspect 16, wherein the coupling mechanism is a magnetic coupling mechanism.
18. The system of aspect 17, wherein the coupling mechanism on the drug-delivery device comprises a first magnet and a release sleeve, and wherein the medication cartridge comprises a second magnet configured to couple with the first magnet.
19. The system of any of aspects 17-18, wherein the coupling mechanism is configured to allow a user to decouple the medication cartridge from the drug-delivery device by moving the release sleeve.
20. The system of aspect 19, wherein the release sleeve comprises a hollow cylindrical body and one or more tabs that extend into an interior volume of the cylindrical body, wherein when the user moves the release sleeve in a proximal direction, the one or more tabs exert a proximal force on a distal end of the medication cartridge to overcome an attractive magnetic force between the first magnet and the second magnet.
21. A method for using a drug-delivery device and a recharging and medication storage hub for the drug-delivery device, the hub having a port, a charging station, a medication storage chamber in alignment with the port, and a medication cartridge storing a medication disposed within the medication storage chamber, the method comprising: placing at least part of the drug-delivery device in proximity with the charging station to charge a rechargeable power source mounted on the drug-delivery device; removing the drug-delivery device from the charging station; inserting at least part of the drug-delivery device through the port and into the medication storage chamber to couple the drug-delivery device with the medication cartridge stored within the medication storage chamber; and removing the drug delivery device from the port once coupled with the medication cartridge so as to remove the medication cartridge from the medication storage chamber.
22. The method of aspect 21 wherein a Quick Response (QR) code is disposed on the hub, the QR code containing information regarding the medication.
23. The method of any of aspects 21-22 wherein the hub provides an indication to a user that the medication cartridge is ready for use via at least one of an audio speaker disposed on the hub, one or more visual indicators disposed on the hub, and a wireless transmission to a mobile device.
24. The method of any of aspects 21-23 wherein the port is covered by a door, and the hub is controlled via a smartphone, such that a user may instruct the hub to open, close, lock, or unlock the door.
25. The method of any of aspects 21-23 wherein the hub is controlled via a smartphone and the hub further comprises a temperature-changing device configured to cool an interior temperature of the medication storage chamber to a storage temperature, the method further comprising instructing the hub via the smartphone to raise the interior temperature of the medication storage chamber using the temperature-changing device.
26. The method of any of aspects 21-25, further comprising re-inserting the at least part of the drug-delivery device through the port and into the medication storage chamber after activating the drug-delivery device to administer the medication, and releasing the medication cartridge coupled with the drug-delivery device into the medication storage chamber for disposal.
27. The method of any of aspects 21-26 wherein the medication cartridge is a first medication cartridge of a plurality of medication cartridges, the medication storage chamber is a first medication storage chamber of a plurality of medication storage chambers defined within a bin disposed within the hub, and each medication storage chamber of the plurality of medication storage chambers is configured to store one medication cartridge of the plurality of medication cartridges, the method further comprising: after removing the drug-delivery device from the port once coupled with the first medication cartridge so as to remove the first medication cartridge from the first medication storage chamber, moving the bin to position another of the plurality of medication storage chambers in alignment with the port.
28. A method for using a recharging and medication storage hub system in conjunction with a mobile device, comprising: providing the hub system, the hub system comprising: a drug-delivery device having a rechargeable power source, and a hub, the hub comprising: a port having a door configured to at least one of (i) move between an open and a closed position and (ii) switch between a locked and an unlocked configuration, a charging station configured to charge the rechargeable power source of the drug-delivery device when at least a portion of the drug-delivery device is placed in proximity with the charging station, a medication storage chamber, and a medication cartridge storing a medication disposed within the medication storage chamber; establishing a communication link between the hub and the mobile device; sending, from the mobile device, an instruction to the hub to prepare the medication cartridge for administration, wherein the instruction causes the hub to move the door to the open position or switch the door to the unlocked configuration to allow access to the medication cartridge; removing the drug-delivery device from the charging station; inserting the drug-delivery device into the port to couple the drug-delivery device with the medication cartridge; and removing the drug-delivery device from the port once coupled with the medication cartridge so as to remove the medication cartridge from the medication storage chamber.
29. The method of aspect 28 further comprising disposing of the medication cartridge by inserting the drug-delivery device into the port, decoupling the medication cartridge from the drug-delivery device, and depositing the medication cartridge into the medication storage chamber.
30. The method of aspect any of aspects 28-29, wherein the hub further comprises a temperature-changing device configured to control an interior temperature of the medication storage chamber, the method further comprising: cooling the interior temperature of the medication storage chamber to a storage temperature using the temperature-changing device; in response to the received instruction, raising the interior temperature of the medication storage chamber to an administration temperature using the temperature-changing device; and providing an indication to a user only after the interior temperature of the medication storage chamber has reached the administration temperature.
31. The method of any of aspects 28-30, wherein the indication may be issued via at least one of one or more audible sounds generated from at least one audio speaker disposed on the hub, one or more visual indicators disposed on the hub, and one or more wireless transmission from the hub to the mobile device.

1. A recharging and medication storage hub for a drug-delivery device, the hub comprising:
a housing defining a port sized to receive at least part of the drug-delivery device;
a charging station disposed on the housing configured to charge a rechargeable power source mounted on the drug-delivery device when at least part of the drug-delivery device is positioned in proximity with the charging station; and
a medication storage chamber configured to align with the port, the medication storage chamber configured to store a medication cartridge;
wherein when at least part of the drug-delivery device is inserted through the port and into the medication storage chamber and then withdrawn from the medication storage chamber, the medication storage chamber is configured to allow the drug-delivery device to withdraw the medication cartridge from the medication storage chamber.

2. The hub of claim 1, wherein the medication cartridge comprises a syringe having a barrel storing a medication, a needle, and a movable plunger.

3. The hub of claim 1, further comprising the medication cartridge, the medication cartridge storing a medication.

4. The hub of claim 1, wherein:
the drug-delivery device comprises a proximal end and a distal end; and
the port is configured to receive at least the proximal end of the drug-delivery device.

5. The hub of claim 1, further comprising a cooling device configured to lower an interior temperature of the medication storage chamber.

6. The hub of claim 1, further comprising a heating device configured to raise an interior temperature of the medication storage chamber.

7. The hub of claim 1, further comprising a temperature-changing device configured to both lower and raise an interior temperature of the medication storage chamber.

8. The hub of claim 1, wherein:
the medication cartridge is a first medication cartridge of a plurality of medication cartridges,
the medication storage chamber is a first medication storage chamber of a plurality of medication storage chambers defined within a bin disposed at least partially within the housing; and
each medication storage chamber of the plurality of medication storage chambers is configured to store one medication cartridge of the plurality of medication cartridges.

9. The hub of claim 8, wherein the bin is movable so as to position only one of the plurality of medication storage chambers in alignment with the port at a time.

10. The hub of claim 9, wherein the bin is configured to rotate so as to position only one of the plurality of medication storage chambers in alignment with the port at a time.

11. The hub of claim 1, wherein:
the port is a first port of a plurality of ports defined in the housing, each port sized to receive at least part of the drug-delivery device;
the medication cartridge is a first medication cartridge of a plurality of medication cartridges; and
the medication storage chamber is a first medication storage chamber of a plurality of medication storage chambers, each medication storage chamber disposed in alignment with a separate port of the plurality of ports, and each medication storage chamber being configured to store one medication cartridge of the plurality of medication cartridges.

12. The hub of claim 1, wherein the medication storage chamber is configured to store the medication cartridge after the medication cartridge is used and de-coupled with the drug-delivery device.

13. The hub of claim 1, wherein the hub further comprises:
a communication device; and
a controller configured to:
receive, via the communication device, data recorded by sensors mounted on the drug-delivery device; and
send, via the communication device, information based on such recorded data to a remote server.

14. The hub of claim 1, wherein the hub further comprises:
one or more sensors configured to record data indicative of a status of the hub;
a communication device; and
a controller configured to send, via the communication device, data based on said recorded data to a remote server.

15. A system for delivering and storing medication, the system comprising the recharging and medication storage hub and the drug-delivery device of claim 1.

16. The system of claim 15, wherein the drug-delivery device comprises a coupling mechanism configured to releasably couple with the medication cartridge.

17. The system of claim 16, wherein the coupling mechanism is a magnetic coupling mechanism.

18. The system of claim 17, wherein the coupling mechanism on the drug-delivery device comprises a first magnet and a release sleeve, and wherein the medication cartridge comprises a second magnet configured to couple with the first magnet.

19. The system of claim 18, wherein the coupling mechanism is configured to allow a user to decouple the medication cartridge from the drug-delivery device by moving the release sleeve.

20. The system of claim 19, wherein the release sleeve comprises a hollow cylindrical body and one or more tabs that extend into an interior volume of the cylindrical body, wherein when the user moves the release sleeve in a proximal direction, the one or more tabs exert a proximal force on a distal end of the medication cartridge to overcome an attractive magnetic force between the first magnet and the second magnet.

21. A method for using a drug-delivery device and a recharging and medication storage hub for the drug-delivery device, the hub having a port, a charging station, a medication storage chamber in alignment with the port, and a medication cartridge storing a medication disposed within the medication storage chamber, the method comprising:
placing at least part of the drug-delivery device in proximity with the charging station to charge a rechargeable power source mounted on the drug-delivery device;
removing the drug-delivery device from the charging station;
inserting at least part of the drug-delivery device through the port and into the medication storage chamber to couple the drug-delivery device with the medication cartridge stored within the medication storage chamber; and
removing the drug delivery device from the port once coupled with the medication cartridge so as to remove the medication cartridge from the medication storage chamber.

22. The method of claim 21 wherein a Quick Response (QR) code is disposed on the hub, the QR code containing information regarding the medication.

23. The method of claim 21 wherein the hub provides an indication to a user that the medication cartridge is ready for use via at least one of an audio speaker disposed on the hub, one or more visual indicators disposed on the hub, and a wireless transmission to a mobile device.

24. The method of claim 21 wherein the port is covered by a door, and the hub is controlled via a smartphone, such that a user may instruct the hub to open, close, lock, or unlock the door.

25. The method of claim 24 wherein the hub is controlled via a smartphone and the hub further comprises a temperature-changing device configured to cool an interior temperature of the medication storage chamber to a storage temperature, the method further comprising instructing the hub via the smartphone to raise the interior temperature of the medication storage chamber using the temperature-changing device.

26. The method of claim 21, further comprising re-inserting the at least part of the drug-delivery device through the port and into the medication storage chamber after activating the drug-delivery device to administer the medication, and releasing the medication cartridge coupled with the drug-delivery device into the medication storage chamber for disposal.

27. The method of claim 21 wherein the medication cartridge is a first medication cartridge of a plurality of medication cartridges, the medication storage chamber is a first medication storage chamber of a plurality of medication storage chambers defined within a bin disposed within the hub, and each medication storage chamber of the plurality of medication storage chambers is configured to store one medication cartridge of the plurality of medication cartridges, the method further comprising:
after removing the drug-delivery device from the port once coupled with the first medication cartridge so as to remove the first medication cartridge from the first medication storage chamber, moving the bin to position another of the plurality of medication storage chambers in alignment with the port.

28. A method for using a recharging and medication storage hub system in conjunction with a mobile device, comprising:
providing the hub system, the hub system comprising:
a drug-delivery device having a rechargeable power source, and
a hub, the hub comprising:
a port having a door configured to at least one of (i) move between an open and a closed position and (ii) switch between a locked and an unlocked configuration,
a charging station configured to charge the rechargeable power source of the drug-delivery device when at least a portion of the drug-delivery device is placed in proximity with the charging station,
a medication storage chamber, and
a medication cartridge storing a medication disposed within the medication storage chamber;
establishing a communication link between the hub and the mobile device;
sending, from the mobile device, an instruction to the hub to prepare the medication cartridge for administration, wherein the instruction causes the hub to move the door to the open position or switch the door to the unlocked configuration to allow access to the medication cartridge;
removing the drug-delivery device from the charging station;
inserting the drug-delivery device into the port to couple the drug-delivery device with the medication cartridge; and
removing the drug-delivery device from the port once coupled with the medication cartridge so as to remove the medication cartridge from the medication storage chamber.

29. The method of claim 28 further comprising disposing of the medication cartridge by inserting the drug-delivery device into the port, decoupling the medication cartridge from the drug-delivery device, and depositing the medication cartridge into the medication storage chamber.

30. The method of claim 28, wherein
the hub further comprises a temperature-changing device configured to control an interior temperature of the medication storage chamber, the method further comprising:
cooling the interior temperature of the medication storage chamber to a storage temperature using the temperature-changing device;
in response to the instruction sent from the mobile device to the hub, raising the interior temperature of the medication storage chamber to an administration temperature using the temperature-changing device; and
providing an indication to a user only after the interior temperature of the medication storage chamber has reached the administration temperature.

31. The method of claim 28, wherein the indication may be issued via at least one of one or more audible sounds generated from at least one audio speaker disposed on the hub, one or more visual indicators disposed on the hub, and one or more wireless transmission from the hub to the mobile device.
